(19) United States
(12) Patent Application Publication
(10) Pub. No.: US 2025/0134681 A1
(43) Pub. Date: May 1, 2025

(54) CONFORMABLE COVER FOR MEDICAL DEVICES

(71) Applicant: W. L. Gore & Associates, Inc., Newark, DE (US)

(72) Inventors: James D. Silverman, Flagstaff, AZ (US); Tyson J. Skelton, Flagstaff, AZ (US)

(21) Appl. No.: 18/686,593

(22) PCT Filed: Aug. 26, 2022
(86) PCT No.: PCT/US2022/041684

§ 371 Date: Feb. 26, 2024

Related U.S. Application Data
(60) Provisional application No. 63/237,192, filed on Aug. 26, 2021.

(51) Int. Cl. A61F 2/90

(52) U.S. Cl. CPC A61F 2/90; A61F 2210/0076; A61F 2230/0091; A61F 2240/001; A61F 2250/0039

(57) ABSTRACT

An implantable medical device includes a frame including a first structural element having a first diameter and a bias-wrapped membrane including at least a first helically wrapped film and a second helically wrapped film overlapping the first helically wrapped film at an angle, arranged on a first side of the first structural element. The first helically wrapped film has a first plurality of edges having an arcuate shape extending along the first side of the first structural element and the arcuate shape of the first plurality of fibrils extends for a majority of the first structural element side.

### CROSS-REFERENCE TO RELATED APPLICATION

This application is a national phase application of PCT Application No. PCT/US2022/041684, internationally filed on Aug. 26, 2022, which claims the benefit of Provisional Application No. 63/237,192, filed Aug. 26, 2021, which are incorporated herein by reference in their entireties for all purposes.

### FIELD

The present disclosure relates generally to apparatuses, systems, and methods that include conformable covers for medical devices. More specifically, the disclosure relates to apparatuses, systems, and methods that include a conformable cover that is arranged with a medical device having frame elements of differing diameters, pitches, or angles.

### BACKGROUND

Implantable medical devices may include one or more stent components and one or more graft, cover, or membrane components. The one or more stent components may include multiple angles, diameters, or pitches. The one or more graft components, when attached or coupled to the frame components, can inhibit or hinder stent movement.

### SUMMARY

Some examples from this specification address implantable medical devices that include stent and graft components that are attached to each other while remaining conformable without inhibiting or hindering stent movement. Additional features and advantages will be apparent from the description that follows and associated drawings.

In a first example (“Example 1”), an implantable medical device includes a frame including a first structural element having a first side and a second side opposite the first side. The implantable device further includes a bias-wrapped membrane including at least a first helically wrapped film and a second helically wrapped film overlapping the first helically wrapped film at an angle, each arranged on the first side of the first structural element. The first helically wrapped film has a first plurality of fibrils having an arcuate shape extending along the first side of the first structural element. The arcuate shape of the first plurality of fibrils extends for a majority of the first structural element side.

In a second example (“Example 2”), the implantable medical device of Example 1 further includes that the frame includes a second structural element having a first side and a second side opposite the first side and wherein the second structural element has a circumference smaller than a circumference of the first frame element, wherein the first plurality of fibrils of the first helically wrapped film extend along the first side of the second structural element.

In a third example (“Example 3”), the implantable medical device of Example 2 further includes that the second helically wrapped film extends along a second side of the first structural element and the second structural element, and wherein the second helically wrapped film includes a second plurality of fibrils along the second side of the first structural element with an arcuate shape and the second plurality of fibrils extend along the second side of the second structural element.

In a fourth example (“Example 4”), the implantable medical device of Example 3 further includes that the frame includes a third structural element that extends at an angle of between approximately 15 degrees and approximately 85 degrees relative to a longitudinal axis of the frame, wherein the first plurality of fibrils extends along the third structural element side at an angle between approximately 15 degrees and approximately 85 degrees in a helical configuration.

In a fifth example (“Example 5”), the implantable medical device of any one of Examples 1-4 further includes that at least a portion of the first helically wrapped film of the bias-wrapped membrane is coupled to at least part of the second helically wrapped film of the bias-wrapped membrane.

In a sixth example (“Example 6”), the implantable medical device of any one of Examples 1-4 further includes that the first structural element is coupled to at least a portion of the bias-wrapped membrane along at least one of the first side and second side of the first structural element and wherein the second structural element is coupled to at least a portion of the bias-wrapped membrane along at least one of the first side and second side of the second structural element.

In a seventh example (“Example 7”), the implantable medical device of any one of Examples 1-6 further includes that the first plurality of fibrils are configured to slide relative to adjacent arcuate fibrils.

In an eight example (“Example 8”), the implantable medical device of any one of Examples 1-7 further includes that the first structural element is a flange and wherein a sensor is coupled to or integral with the flange and the flange is operable to engage the sensor and tissue for operable engagement between the sensor and the tissue.

In a ninth example (“Example 9”), the implantable medical device of any one of claims 1-7 further includes that the first structural element is a flange and the implantable medical device comprises an occluder coupled to or integral with the flange, and wherein the flange is configured to anchor the occluder to tissue.

In a tenth example (“Example 10”), an implantable medical device includes a frame including a first frame element having a first circumference and a second frame element having a second circumference, the first frame element having a first side and a second side opposite the first side, and a bias-wrapped membrane, including at least a first helically wrapped film and a second helically wrapped film overlapping the first helically wrapped film at an angle. The bias-wrapped membrane is arranged on the first side of the first frame element and the bias-wrapped membrane has a first plurality of fibrils arranged in an arcuate shape along the first side of the first frame element and a second plurality of fibrils arranged in an arcuate shape along the first side of the second frame element. The bias-wrapped membrane has a third plurality of fibrils arranged between the first and second plurality of fibrils, wherein the third plurality of fibrils have a pitch angle.

In an eleventh example (“Example 11”), a method of making an implantable medical device that includes a frame including a first frame element having a first side opposite a second side and a first diameter and a second frame element having a first side opposite a second side and having a second diameter, the first diameter being different than the second diameter, includes elongating a first bias-wrapped membrane including a first helically wrapped film and second helically wrapped film overlapping the first helically wrapped film at an angle, so as to alter a first wrap angle of a first plurality of fibrils of the first bias-wrapped membrane. The method further includes arranging the elongated first bias-wrapped membrane on the first side of the first frame element and the second frame element so as to define a first bias-wrapped membrane first edge along the first frame element and an arcuate shaped bias wrapped film edge along the first frame element. The method further includes elongating a second-bias wrapped membrane, including a first helically wrapped film and second helically wrapped film overlapping the first helically wrapped film at an angle, to alter a second wrap angle of a second plurality of fibrils of the second bias-wrapped membrane. The method further includes arranging the elongated second-bias-wrapped membrane on the second side of the first frame element and the second frame element to define a second-bias-wrapped membrane first edge along the first frame element, wherein the second-bias-wrapped membrane first edge has an arcuate shape.

In a twelfth example (“Example 12”), the method of Example 11 further includes coupling at least a part of the first bias-wrapped membrane to at least a part of the second bias-wrapped membrane.

In a thirteenth example (“Example 13”), the method of Example 11 further includes coupling at least a part of the first bias-wrapped membrane to at least a part of the first frame element and the second frame element, and coupling at least a part of the second bias-wrapped membrane to at least a part of the first frame element and the second frame element.

In a fourteenth example (“Example 14”), the method of any of Examples 12-13 further includes that the first plurality of fibrils are configured to slide relative to the second plurality of fibrils, and the first bias-wrapped membrane and the second bias-wrapped membrane are configured to maintain contact with the first frame element and the second frame element.

In a fifteenth example (“Example 15”), the method of any one of Examples 12-14 further includes that the pitch angle of the first plurality of fibrils is a first pitch angle in the bias-wrapped membrane that is aligned with the first frame element and a second pitch angle in the bias-wrapped membrane that is aligned with the second frame element, wherein the first pitch angle is different than the second pitch angle.

In a sixteenth example (“Example 16”), the method of Example 15 further includes that the pitch angle of the second plurality of fibrils includes a third pitch angle in the bias-wrapped membrane that is aligned with the first frame element and a fourth pitch angle in the bias-wrapped membrane that is aligned with the second frame element, wherein the third pitch angle is different than the fourth pitch angle.

In a seventeenth example (“Example 17”), the method of Example 16 further includes that at least one of the second pitch angle and the fourth pitch angle is between approximately 15 degrees and approximately 85 degrees.

In an eighteenth example (“Example 18”), a method of making an implantable medical device includes stretching a first bias-wrapped membrane in a first direction along a mandrel, the mandrel defining a first section and a flange section, applying a first frame element to a first side of the flange section of the mandrel, stretching a second bias-wrapped membrane in a second direction that is opposite the first direction along the mandrel so as to cover the first frame element, and everting the second bias-wrapped membrane in the first direction along the mandrel. The method further includes applying a second frame element to the first side of the flange section of the mandrel and everting the second bias-wrapped membrane in the second direction along the mandrel so as to sandwich the first frame element and the second frame element between the first bias-wrapped membrane and the second bias-wrapped membrane.

In a nineteenth example (“Example 19”), a method of making an implantable medical device that includes a frame including a first frame element, a second frame element, and a third frame element, includes elongating a first bias-wrapped membrane including a first helically wrapped film and second helically wrapped film overlapping the first helically wrapped film at an angle, so as to alter a first wrap angle of a first plurality of fibrils of the first bias-wrapped membrane. The method further includes arranging the elongated first bias-wrapped membrane on a first side of the first frame element and the second frame element and the third frame element so as to define a first pitch angle along the second frame element and a plurality of arcuate shaped fibrils extending along a majority of the first side of the first frame element and the second frame element. The method further includes elongating a second-bias wrapped membrane, including a first helically wrapped film and second helically wrapped film overlapping the first helically wrapped film at an angle, to alter a second wrap angle of a second plurality of fibrils of the second bias-wrapped membrane and arranging the elongated second-bias-wrapped membrane on the second side of the first frame element and the second frame element and the third frame element to define a second-bias-wrapped membrane first fibril orientation along the second frame element and a second-bias-wrapped membrane second fibril orientation along the first frame element and the second frame element, wherein the second-bias-wrapped membrane has plurality of first fibrils orientated in an arcuate shape along a majority of the first frame element second side and the third frame element second side.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an illustration on of an example implantable medical device in accordance with an embodiment;

FIG. 2 is an illustration of a portion of an example implantable medical device with one or more bias-wrapped membranes in accordance with an embodiment;

FIG. 3A is an illustration of an example implantable medical device with one or more bias-wrapped membranes in accordance with an embodiment;

FIG. 3B is a cross-sectional illustration of the implantable medical device, shown in FIG. 3A, in accordance with an embodiment;

FIG. 4A is a photograph of an example implantable medical device in an example first step of a method of making the implantable medical device;

FIG. 4B is schematic illustration of the example implantable medical device of FIG. 4A;

FIG. 5A is a photograph of the implantable medical device, shown in FIG. 4A, in an example second step of a method of making the implantable medical device;

FIG. 5B is a schematic illustration of the example implantable medical device of FIG. 5A;

FIG. 5C is a cross-section of the implantable medical device as shown in FIG. 5B, in the example second step of a method of making the implantable medical device;

FIG. 6A is a photograph of the implantable medical device, shown in FIGS. 4A and 5A, in an example third step of a method of making the implantable medical device;

FIG. 6B is a schematic illustration of the implantable medical device of FIG. 6A.

FIG. 6C is cross-section of the implantable medical device as shown in FIG. 6B, in the example third step of a method of making the implantable medical device;

FIG. 7A is a photograph of the implantable medical device, shown in FIGS. 4A, 5A, and 6A, in an example fourth step of a method of making the implantable medical device; and

FIG. 7B is an illustration of the implantable medical device shown in FIG. 7A.

FIG. 7C is a cross-section the implantable medical device, shown in FIG. 7B, in the example fourth step of a method of making the implantable medical device.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

As used herein, the term “fibril” or “fibrils” refer to small and/or slender fibers forming part of a material layer (e.g., of a membrane). A typical example of a material including “fibrils” includes ePTFE, or expanded polytetrafluoroethylene, which may include a fibril structure with nodes interspersed between fibrils or a fibril structure with the absence, or substantial absence, of nodes. Other materials, e.g., expanded polymeric materials, may also define fibrils, or fibrillated structures. In the figures, fibrils represented by larger, visible lines for visualization purposes in order to convey the bulk orientation, or directionality in which fibrils of layers extend. In various examples, however, the fibrils are actually visible on the microscopic level, and are not generally visible with the naked eye alone. Additionally, while the representation of the fibrils in the figures are meant to convey the bulk orientation, or overall directionality in which the fibrils of layers extend, there may be some fibrils which do not generally align with this bulk orientation.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various aspects of the present disclosure are directed toward implantable medical devices that may include a stent, or frame, and one or more membrane components. The implantable medical device, are more particularly the frame, or frame elements, may include different diameters, multiple angles, or pitches. The tubular membrane, alternatively referred to as membrane, can be coupled or attached to one or more portions of the frame or frame element to form the implantable medical device. For reference, frame elements are often braided, wound, or cut (e.g., laser cut) to define an overall supportive framework for a device. The frame is often times deformable and/or flexible. In various examples, the membrane is formed from one or more helically wrapped films or layers of film. The membrane (or membrane layers) may be arranged with respect to the frame, in a conformable construct such that discontinuities that may arise due to the different frame diameters along a length of the device, multiple frame element angles, or frame element pitches are lessened or eliminated. This conformability or adaptability to frame features may help facilitate, for example, frame deformation, flexibility, and/or conformability depending upon the particular frame characteristics. In certain instances, the membrane or membrane layers may each be a single tube that includes oriented fibrils configured to lessen or eliminate discontinuities that may arise due to the different diameters, multiple angles, or pitches between portions of the frame or frame elements. More particularly, the film that defines the membrane or each membrane layer may be composed of a fibrillated microstructure which may include a plurality of fibrils that may be oriented in a lengthwise, or elongated direction, to define the helically wrapped film or layers of film. The membrane or membrane layers may conform to the shape of the frame or frame elements as described in further detail below.

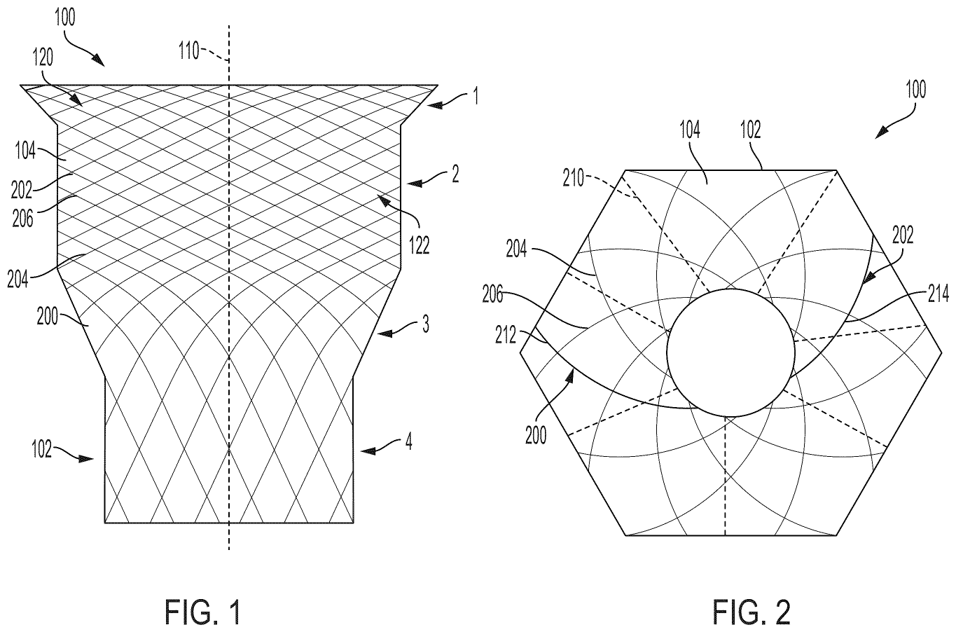

FIG. 1 is a side view of an example implantable medical device 100 in accordance with an embodiment. The implantable medical device 100 may include a frame 102 (e.g., a stent). The frame 102 (and implantable medical device 100) may include multiple diameters, pitches, and/or changes in shape. As shown, the frame 102, for example, includes at least four different diameters, pitches, and changes in shape. In certain instances, one of the areas of the frame 102 may include a flange (portion 1) that extends at an angle outwardly relative to another portion (portion 2) of the frame 102. The flange may be used as an anchor and/or fixation portion of the implantable medical device 100 for coupling to a target location within a patient. The flange (portion 1) of the frame 102 may be perpendicular to the other portion (portion 2) of the frame 102 in certain instances. As shown, the frame 102 includes portion 1 (e.g., a first portion), portion 2 (e.g., a second portion), portion 3 (e.g., a third portion), and portion 4 (e.g., a fourth portion), each of which may include different diameters, slopes, pitches, or angles relative to a longitudinal axis 110 of the frame 102. In certain examples, the implantable medical device 100 may include, or otherwise be configured as, an occluder, a sensor, an appendage isolator, a shunt, or other implantable device. In other words, various devices may be incorporated with, defined by, or otherwise associated with, the implantable medical device 100 and the present disclosure is not meant to be limited by the above examples. In various embodiments, the flange may be used to couple a sensor, appendage isolator, shunt, or other suitable device, to a target location within the patient to allow for desired operation of the device. For example, the flange may operably couple or engage the tissue of the target location with a sensor to allow for monitoring, treatment delivery, or various other functions to be completed. As another example, the flange may anchor an occluder to the tissue of a target location to ensure proper fixation of the occluder to the tissue.

In certain instances, and as described in further detail below, the frame 102 may include a membrane or membranes 104 coupled or attached to frame 102. The membrane or membranes 104 may be configured to conform and/or align with the individual sections of the frame 102 that have varying diameters, pitches, or changes in shape as explained in further detail below. The implantable medical device 100 may be a heart valve, shunt, occluder, stent, or other implantable medical device that includes diameters, pitches, or changes in shape as noted above.

As shown in FIG. 1, the frame 102 is covered by the membrane 104. The membrane 104 is a bias-wrapped membrane 104 that may include a first helically wrapped film 200 (represented generally by lines designating a first plurality of fibrils 204 and the direction in which those fibrils are oriented) having a first helically wrapped film edge 212 (FIG. 2), and a second helically wrapped film 202 (represented generally by lines designating a second plurality of fibrils 206 and the orientation in which those fibrils are oriented) having a second helically wrapped film edge 214 (FIG. 2). The first helically wrapped film 200 and the second helically wrapped film 202 may form separate membranes (i.e., more than one membrane 104) or may be attached or coupled together to form a single membrane 104 as noted above. Additionally, the first plurality of fibrils 204 and the second plurality of fibrils 206 are illustrated schematically herein to denote the directionality of the first and second plurality of fibrils 204, 206 within the first and second helically wrapped films 200, 202. Further, the term of “bias-wrapped” described herein may be defined by the orientation of the first and second helically wrapped films 200, 202 and more particularly the orientation of the plurality of fibrils 204, 206 of the films 200, 202. However, in some embodiments, at least one of the plurality of fibrils may not align with the frame 102 or have generally the same orientation or bulk directionality as a majority of the plurality of fibrils. Due to the random nature of the fibrils in how the fibrils extend from nodes, it may the case that various of the fibrils will extend from the nodes in directions that are not generally aligned with the overall orientation of the first and second helically wrapped films 200, 202. Therefore, the first plurality of fibrils 204 and the second plurality of fibrils 206 as shown in the figures herein may represent the majority of the fibrils and not the orientation of each individual fibril that may compose the first and second helically wrapped films 200, 202. In other words, the directionality of the first plurality of fibrils 204 and the second plurality of fibrils 206 as shown in the figures herein may represent the average directionality of each of the fibrils 204, 206. In other instances, the directionality of the first plurality of fibrils 204 and the second plurality of fibrils 206 as shown in the figures may represent the average directionality of a majority of the fibrils 204, 206.

In certain instances, the second helically wrapped film 202 may at least partially overlap the first helically wrapped film 200 at an angle. As noted above, the frame 102 may include multiple diameters, with the frame 102 including multiple structural elements with each portion of the frame 102 defined by, or otherwise including a different structural element.

In certain instances, the first helically wrapped film 200 and the second helically wrapped film 202 are at least partially overlapping at an angle. In addition, the first helically wrapped film 200 is arranged on a first side of a first structural element (portion 1 of the frame 102). In certain instances, the bias-wrapped membrane 104 includes the first plurality of fibrils 204, and consequently includes the first helically wrapped film edge 212, as shown in FIG. 2, thus defining a first bias-wrapped membrane edge of the bias-wrapped membrane 104. In this way, the first helically wrapped film edge 212, and thus the first bias-wrapped membrane edge of the first bias-wrapped membrane 104, have an arcuate shape that extend along the first structural element (portion 1). In embodiments, the first structural element (portion 1) may include a first side 120 and a second side (not shown) that is opposite the first side 120. In certain instances, the first side 120 and the second side may be opposites, or located in an opposing manner relative to one another (e.g., an inside or outside surface of the first structural element (portion 1)). In some embodiments, the first helically wrapped film edge 212, and thus the first bias-wrapped membrane edge of the first bias-wrapped membrane 104, extend along the first side 120 of the first structural element (portion 1 of the frame 102 (FIG. 1)), e.g. in instances where the structural element has a changing circumference, as in portions 1 and 3 of FIG. 1, and as shown in FIG. 2. The arcuate shape of the first plurality of fibrils 204 of the first bias-wrapped membrane 104 extends for a majority of the first side 120 of the first structural element (portion 1), or in some examples, a majority of a structural element. In certain instances, the first plurality of fibrils 204, and consequently the first helically wrapped film edge 212, and the second plurality of fibrils 206, and consequently the second helically wrapped film edge 214, have an arcuate shape along structural elements that have a changing circumference (as shown in FIG. 1, portions 1 and 3). In certain instances, the first plurality of fibrils 204, and consequently the first helically wrapped film edge 212, and the second plurality of fibrils 206, and consequently the second helically wrapped film edge 214, have a helical shape (as shown in FIG. 1, portions 2 and 4).

In certain instances, the frame 102 includes a second structural element (e.g., portion 2 (FIG. 1)) having a first side 122 and a second side (not shown) opposite the first side 122. In certain instances, the first side 122 and the second side may be opposites, or located in an opposing manner relative to one another (e.g., an inside or outside). The second structural element (portion 2) of the frame 102 has a circumference smaller than the circumference of the first frame element. In addition, the first plurality of fibrils 204 of the first helically wrapped film 200, and consequently the first helically wrapped film edge 212, extend along the first side 122 of the second structural element (portion 2). In certain instances, the second helically wrapped film 202 extends along the second side of the first structural element (portion 1 of the frame 102 (FIG. 1)) and the second structural element (portion 2 of the frame 102). In certain instances, the bias wrapped membrane 104 including the first and the second helically wrapped films 200, 202 overlapping each other, extends along the first side 120 and in some instances the first side 122 and the second side of the first structural element (portion 1), and in some instances along the first side of more than the first structural element (portion 1), such as the second structural element (portion 2), and in some instances along the first side and the second side of additional structural elements. As noted above, the second helically wrapped film 202 includes the second plurality of fibrils 206, and the second plurality of fibrils 206 may extend along the second side of the first structural element with an arcuate shape and the second plurality of fibrils 206 extend along the second side (not shown) of the second structural element (portion 2), at an angle between approximately 15 degrees and approximately 85 degrees defining a straightened helical angle. For example, the angle may be between approximately 15 degrees and approximately 80 degrees, between approximately 20 degrees and approximately 75 degrees, between approximately 25 degrees and approximately 65 degrees, between approximately 30 degrees and approximately 50 degrees, or between approximately 35 degrees and approximately 45 degrees.

In certain instances, the frame 102 includes a third structural element (portion 3 of the frame 102 (FIG. 1)) that extends at an angle of between approximately 15 degrees and approximately 85 degrees relative to the longitudinal axis 110 of the frame 102. For example, the angle may be between approximately 15 degrees and approximately 80 degrees, between approximately 20 degrees and approximately 75 degrees, between approximately 25 degrees and approximately 65 degrees, between approximately 30 degrees and approximately 50 degrees, or between approximately 35 degrees and approximately 45 degrees. In certain instances, the arcuate shape of the first plurality of fibrils 204 extend along the third structural element side with an arcuate shape, as shown in FIG. 1.

As noted above, at least a part of the first helically wrapped film 200 of the bias-wrapped membrane 104 is coupled to at least part of the second helically wrapped film 202 of the bias-wrapped membrane 104. In addition, the first structural element (portion 1) is coupled to at least a portion of the bias-wrapped membrane 104 along at least one of the first side 120 and second side of the first structural element (portion 1) and wherein the second structural element (portion 2) is coupled to at least a portion of the bias-wrapped membrane 104 along at least one of the first side 122 and second side of the second structural element (portion 2). In addition, the first plurality of fibrils 204 are configured to slide relative to adjacent arcuate fibrils.

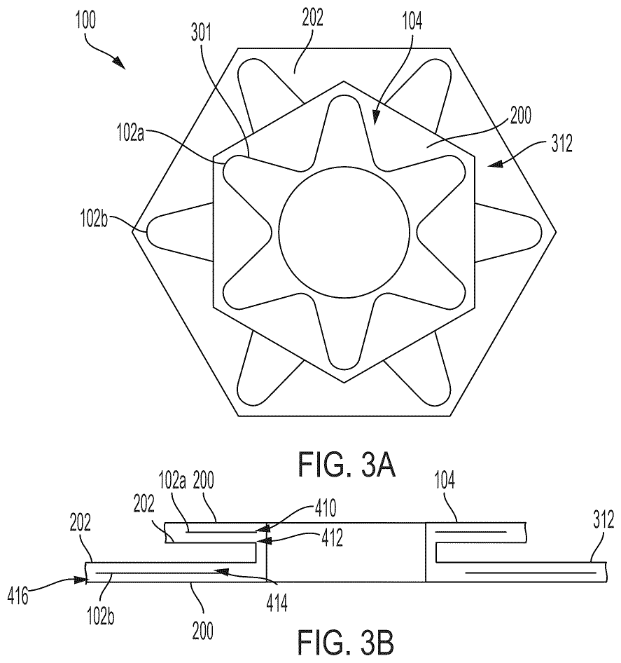

FIG. 2 is an end view illustration of a portion of an example implantable medical device 100 with one or more overlapping first and second pluralities of films 200, 202 that are helically wrapped and form a bias-wrapped membrane 104 in accordance with an embodiment. The implantable medical device 100 may be a heart valve, shunt, occluder, stent, or other implantable medical device. As previously described herein, the first helically wrapped film 200 has the first helically wrapped film first edge 212 and the second helically wrapped film 202 has the second helically wrapped film first edge 214. The first helically wrapped film first edge 212 and the second helically wrapped film first edge 214 intersect in an overlapping fashion due to the overlapping configuration of the first and second helically wrapped films 200, 202. The first helically wrapped film 200 may have the first plurality of fibrils 204 (e.g., as shown in FIG. 1) aligned substantially with the first helically wrapped film first edge 212. The second helically wrapped film 202 may have the second plurality of fibrils 206 (e.g., as shown in FIG. 1) aligned substantially with the second helically wrapped film first edge 214. In certain instances, the implantable medical device 100 includes the frame 102. As shown in FIG. 1 and as shown in further detail in FIG. 3, the frame 102 may include a first frame element 102a having a first diameter and a second frame element 102b having a second diameter, the first diameter being different than the second diameter. As shown in FIG. 2 in phantom and only as an example, a non-bias-wrapped membrane (uniaxial) film edge 210 is shown for comparison.

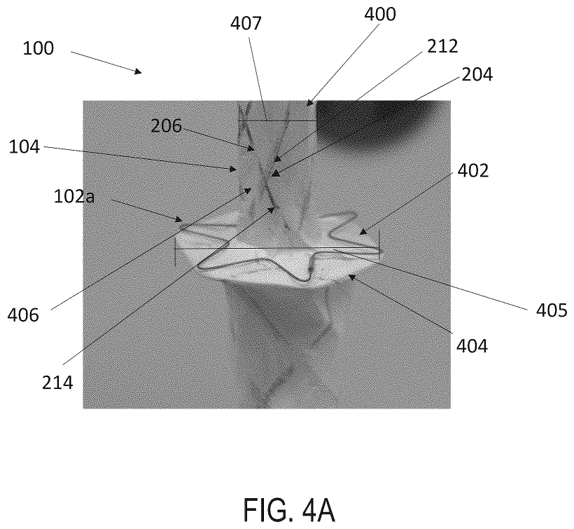

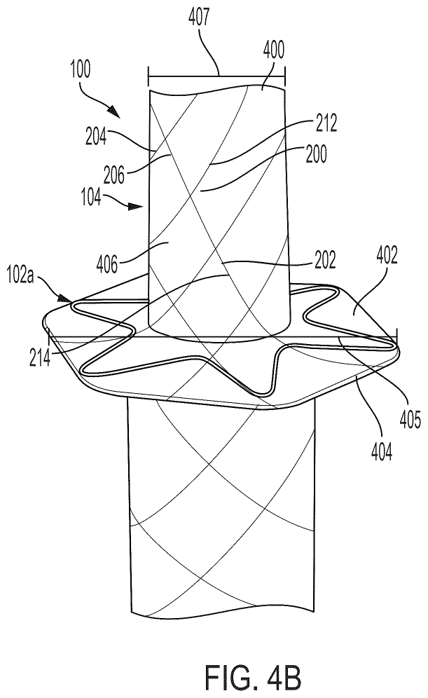

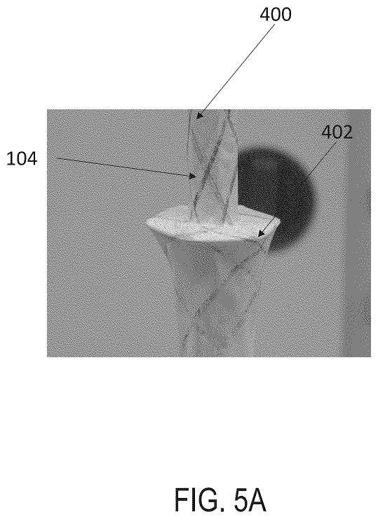

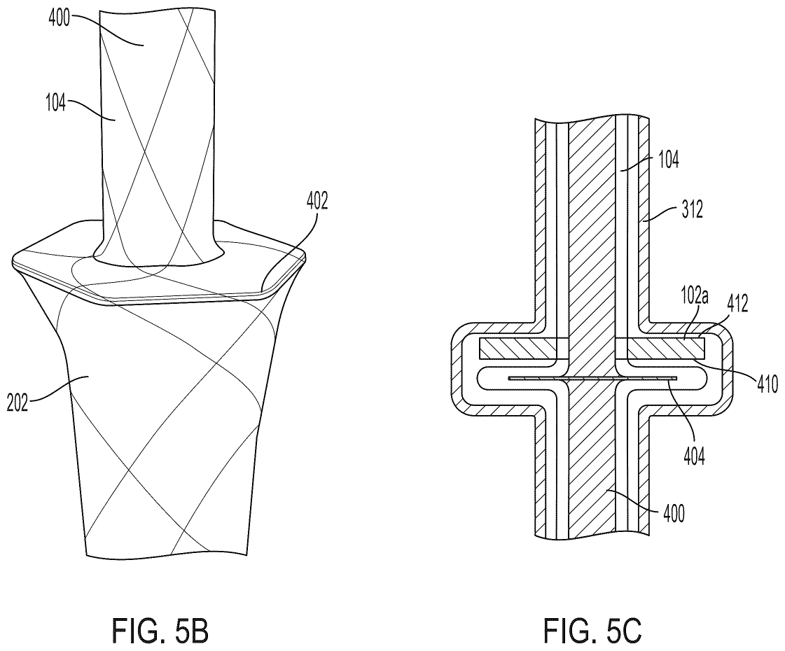

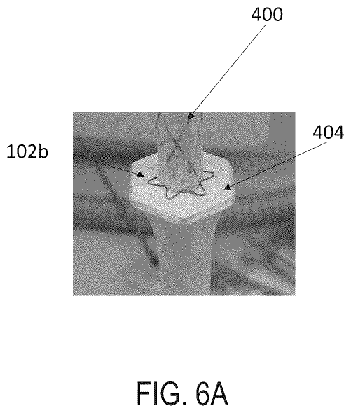

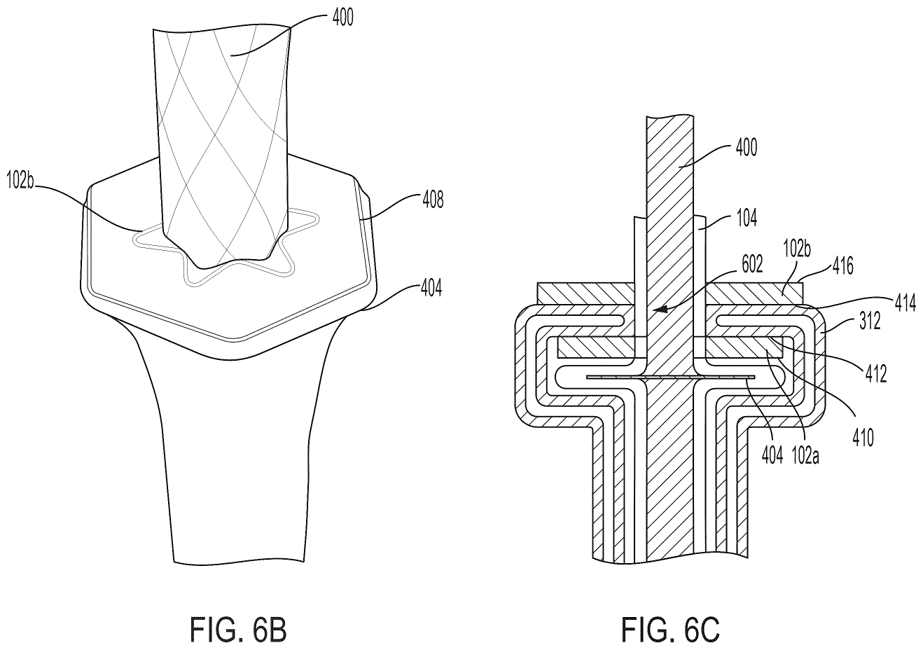

The implantable medical device 100 may also include a first bias-wrapped membrane 104 arranged on the frame 102 (FIG. 4B) and a second bias-wrapped membrane 312 arranged on the frame 102 and the first and second structural elements 102a and 102b (e.g., FIG. 5B and FIGS. 6B, 6C). Similar to the first bias-wrapped membrane 104, the second-bias wrapped membrane 312 may include a first helically wrapped film 200 formed by a first plurality of fibrils 204 and a second helically wrapped film 202 formed by a second plurality of fibrils 206.

In certain instances, the first bias-wrapped membrane 104 may be arranged on a first side of the frame 102 (or first frame element 102a) and the second bias-wrapped membrane 312 may be arranged on a second side of the frame 102 (or first frame element 120a). In certain instances, the first side and the second side may be opposites, or located in an opposing manner relative to one another (e.g., an inside or outside as shown in FIG. 3B), as will be described herein further. In certain instances, the first helically wrapped film 200 includes a first helically wrapped film first edge 212 configured to exhibit a pitch angle aligned with at least a portion of the frame 102, for example in a waist region connecting the first frame element 102a and second frame element 102b (e.g., as shown in FIG. 3A) and for example along a wire portion 301 extending along a face portion of the first frame element 102a and the second frame element 102b. The second helically wrapped film 202 may include the second helically wrapped film first edge 214 that may be configured to exhibit a pitch angle aligned with the frame 102. In certain instances, the first helically wrapped film first edge 212 and the second helically wrapped film first edge 214 are configured to exhibit a pitch angle aligned with the frame 102 at different diameters, pitches, or changes in shape of the frame 102, for example, in a waist region (e.g., as shown in FIG. 3B) connecting two larger diameter portions wherein the helically wrapped films 200, 202 in the two larger diameter portions have an arcuate shape and in waist region the helically wrapped film(s) has a pitch angle aligned with an adjacent structural element (e.g., a wire). In addition, the first helically wrapped film 200 and the second helically wrapped film 202 may also be configured to conform to the frame 102 and slide relatively to one another.

As noted above, in certain instances, the first helically wrapped film first edge 212 and the second helically wrapped film first edge 214 are configured to exhibit a pitch angle aligned with the frame 102, or a portion of the frame 102, at different diameters, pitches, or changes in shape of the frame 102. In some embodiments, the pitch angle may range between approximately 0 degrees and approximately 90 degrees. For example, the pitch angle may range between approximately 5 degrees and approximately 80 degrees, or between approximately 15 degrees and approximately 70 degrees, or between approximately 30 degrees and approximately 50 degrees.

The first helically wrapped film first edge 212 and the second helically wrapped film first edge 214 may be configured to exhibit an angle that changes as a function of diameter, pitch, angle or shape within the frame 102. In other instances, the first helically wrapped film first edge 212 and the second helically wrapped film first edge 214 may be arcuate in certain instances to conform to the different diameters, pitches, or changes in shape of the frame 102 (e.g., as shown in FIG. 3A).

FIG. 3A is an end view of an example implantable medical device 100 with one or more first and second helically wrapped films formed into a tubular membrane and coupled to the first frame element 102a and the second frame element 102b. As illustrated, the first and second frame elements 102a and 102b have different circumferences. However, in other embodiments, the first and second frame elements 102a, 102b may have circumferences that are approximately equal. The implantable medical device 100 may include the frame 102 having the first frame element 102a that includes a first diameter and the second frame element 102b that includes a second diameter. In certain instances, the first diameter is different (e.g., larger or smaller) than the second diameter. However, in other embodiments, the first diameter may be approximately equal to the second diameter.

The implantable medical device 100 may also include the first bias-wrapped membrane 104 and a second bias-wrapped membrane 312. Alternatively the first bias-wrapped membrane 104 may extend along its length and form a second portion of the first bias-wrapped membrane 104, such that the first bias-wrapped membrane 104 includes a first portion and a second portion. FIG. 3B is a simplified cross-sectional illustration of the implantable medical device, shown in FIG. 3A, in accordance with an embodiment. As shown, the first bias-wrapped membrane 104 may be arranged on a first side 410 of the first frame element 102a and a first side 416 of the second frame element 102b and the second bias-wrapped membrane 312 may be arranged on a second side 412 of the first frame element 102a and a second side 414 of the second frame element 102b. In the embodiments wherein the first bias-wrapped membrane 104 includes a first portion and a second portion, the first portion may be arranged on the first side 410 of the first frame element 102a and the first side 416 of the second frame element 102b and the second portion may be arranged on the second side 412 of the first frame element 102a and the second side 414 of the second frame element 102b.

The first bias-wrapped membrane 104 and the second bias-wrapped membrane 312 may be connected, coupled or adhere to one another at one or locations. In certain instances, at least a portion of the first bias-wrapped membrane 104 is bonded to at least a portion of the second bias-wrapped membrane 312. In addition, and in certain instances, at least a portion of the first bias-wrapped membrane 104 is bonded to at least a portion of the first frame element 102a and the second frame element 102b, and at least a portion of the second bias-wrapped membrane 312 is bonded to at least a portion of the first frame element 102a and the second frame element 102b.

In certain instances, the first helically wrapped film 200 may be configured to slide relative to the second helically wrapped film 202 that overlaps the first helically wrapped film 200. In other instances, the first helically wrapped film 200 and the overlapping second helically wrapped film 202 have fibrils respectively, that slide relative to each other within each respective film, that allow an arcuate shape of the helically wrapped films 200, 202 to occur. In addition, the first bias-wrapped membrane 104 and the second bias-wrapped membrane 312 are configured to maintain contact with the first frame element 102a and the second frame element 102b. The first and second frame elements 102a, 102b each may include a plurality of strut elements. The plurality of strut elements may form a first flange 404 that includes the first frame element 102a and a cylindrical fluid passage that includes the second frame element 102b.

FIGS. 4-7 illustrate steps in example methods of making an implantable medical device, consistent with various aspects of the present disclosure. In certain instances, FIGS. 4-7 illustrate steps in example methods of making an implantable medical device 100 that includes a frame 102 including a first frame element 102a having a first diameter and a second frame element 102b having a second diameter with the first diameter being different than the second diameter.

FIG. 4A is a photograph of an example implantable medical device 100 in an example first step of a method of making the implantable medical device. FIG. 4B is a schematic illustration of the implantable medical device 100 of FIG. 4A. A first bias-wrapped membrane 104 is arranged in a tubular form, on a mandrel 400, having a diameter approximately the same as, or slightly less than, as an effective diameter 405 of a flange 404. In certain instances, the first bias-wrapped membrane 104, or a portion of the first bias-wrapped membrane 104 is stretched or elongated to alter a wrap angle of at least the first helically wrapped film 202 of the first bias-wrapped membrane 104 such that the elongated portion has a smaller diameter, for example a diameter approximately equal to the diameter of the mandrel 400. In certain instances, the first bias-wrapped membrane 104, having a first diameter approximately equal to or slightly less than an effective diameter 405 of a flange 404 (shown in a hexagon shape in FIG. 4), is stretched in a first direction along a mandrel 400, forming a first section 402 along a face of the flange 404, and a second section 406 with a second diameter 407, e.g. along mandrel 400, the second diameter 407 having a value of less than the effective diameter D5 of the flange 404.

The stretched, first bias-wrapped membrane 104 is then arranged on a first side 410 of the first frame element 102a. In certain instances, the first frame element 102a is arranged on a first side of the first section 402 of the flange 404 of the mandrel 400. More particularly, the first frame element 102a is positioned onto the first bias-wrapped membrane 104 positioned on the first section 402 of the flange 404 of the mandrel 400 such that the first frame element 102a is arranged around the mandrel 400 and on top of the flange 404. In certain instances, the first section 402 of the flange 404 of the mandrel 400 may include a shape corresponding to the shape of the first frame element 102a to hold the first frame element 102a in a desired location. As discussed in further detail below, the stretched first bias-wrapped membrane 104 is also arranged on the second frame element 102b. As a result, the stretched first bias-wrapped membrane 104, including the first helically wrapped film first edge 212, exhibits an arcuate shape along the first frame element 102a face and the second frame element 102b face.

FIG. 5A is a photograph of the implantable medical device 100 shown in FIG. 4A, in an example second step of a method of making the implantable medical device 100. FIGS. 5B-5C are schematic illustrations of the implantable medical device 100 of FIG. 5A. More particularly FIG. 5B is a side perspective view of the implantable medical device while FIG. 5C illustrates a simplified cross-sectional view of the implantable medical device. A second bias-wrapped membrane 312 is arranged with the first frame element 102a. In certain instances, the second bias-wrapped membrane 312 is stretched to alter a wrap angle of a second plurality of fibrils 206, and alter an edge of at least one of the first and second helically wrapped films 200, 202, of the second bias-wrapped membrane 312. In certain instances, the elongated second bias-wrapped membrane 312 is arranged on the second side 412 of the first frame element 102a and the second side 414 of the second frame element 102b to exhibit an arcuate shape along the first frame element 102a face and the second frame element 102b face. In certain instances, the first helically wrapped film 200 and the second helically wrapped film 202 overlapping the first helically wrapped film 200 of the bias-wrapped membrane 104 are configured to conform to the first frame element 102a and the second frame element 102b. The first helically wrapped film first edge 212 and the second helically wrapped film first edge 214 have an arcuate shape along the first frame element 102a and furthermore the first helically wrapped film first edge 212 and the second helically wrapped film first edge 214 may slide relative to one another. In other embodiments, the first helically wrapped film first edge 212 and the second helically wrapped film first edge 214 do not slide relative to each other. In some embodiments, the fibrils arranged along the first frame element 102a and the second frame element 102b, slide relative to each other such that the first helically wrapped film first edge 212 and the second helically wrapped film first edge 214 form an arcuate shape along their respective edges. The flange 404 is thus conformable to a flat and/or curved surface and acts as an anchor or a fixation element to secure and couple the implantable medical device 100 to surfaces of various locations of a patient, such as but not limited to the stomach, liver, heart, or blood vessels, for example. However, various other surface configurations of the flange 404 may be incorporated to allow for the flange 404 to act as an anchor.

In certain instances, the second bias-wrapped membrane 312 is elongated in a second direction, opposite the first direction, along the mandrel 400 to cover the first frame element 102a, as shown in FIG. 6C.

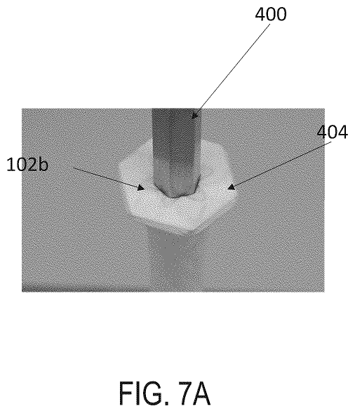

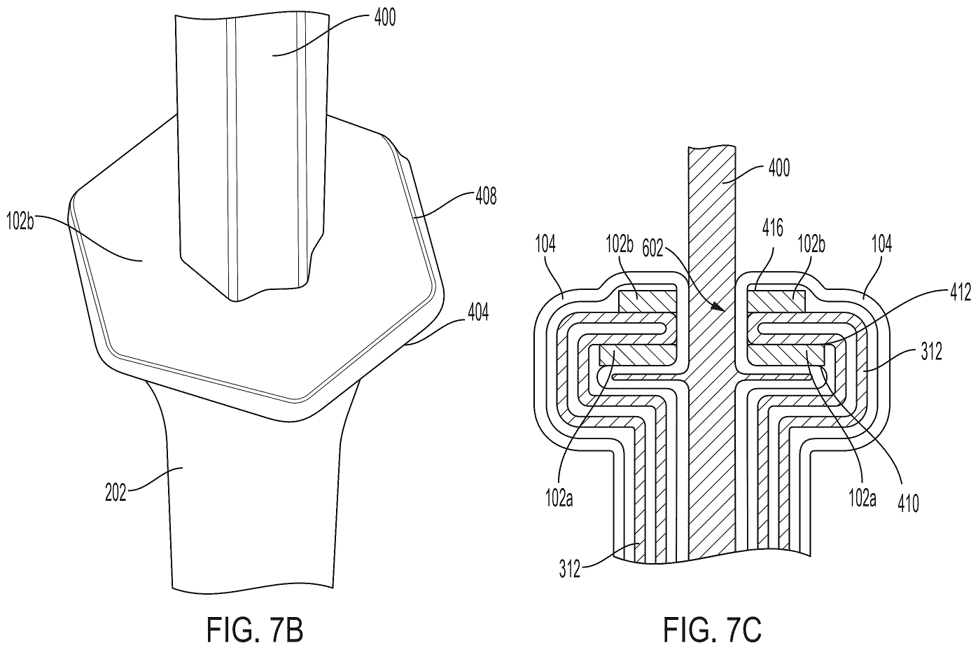

In certain instances, the pitch angle of the first plurality of fibrils, and the first bias-wrapped membrane first edge 212, of the first bias-wrapped membrane 104 includes a first pitch angle in portions of the first bias-wrapped membrane 104 that are aligned with the first frame element 102a and a second pitch angle in portions of the first bias-wrapped membrane 104 that are aligned with the second frame element 102b. The first pitch and the second pitch angle of the first bias-wrapped membrane 104 may be different from one another (e.g., larger or smaller). For example, in some embodiments, the first pitch angle may be larger than the second pitch angle. In other embodiments, the first pitch angle may be smaller than the second pitch angle. The different pitch angles within the first bias-wrapped membrane 204 vary with the different diameters of the first and second frame elements 102a, 102b. The different pitch angles within the first bias-wrapped membrane 204 varying with the different diameters of the first and second frame elements 102, 102b may facilitate the first bias-wrapped membrane 204 conforming with each of the first and second frame elements 102, 102b. The first bias-wrapped membrane, may have arcuate shaped film edges and fibrils along tapered, or angled portions, of implantable medical device 100. For example, in FIG. 1, the first frame element 102a and the second frame element 102b may have pitch angles, that may or may not be aligned with frame elements, and tapered portions may have arcuate shaped film edges and fibrils. In other instances, the pitch angles of the first and second bias-wrapped membranes 104, 312 may align with a first frame element and a second frame element wherein at least one of the first and second frame elements extend along a waist region 602 (as shown in FIGS. 6C and 7C).

In certain instances, (for example the instance of the device shown in FIG. 1), the pitch angle of the second plurality of fibrils, and edge, of the first and second bias-wrapped membrane 104, 312 includes a third pitch angle in portions of the second bias-wrapped membrane 312 that are aligned with the first frame element 102a and a fourth pitch angle in portions of the first and second bias-wrapped membrane 104, 312 that are aligned with the second frame element 102b. In addition, the third pitch angle may be different than the fourth pitch angle. For example, the third pitch angle may have a value that is larger than a value of the fourth pitch angle. Further, in other embodiments, the value of the third pitch angle may be less than the value of the fourth pitch angle. The different pitch angles within the first and second bias-wrapped membranes 104, 312 varying with the different diameters of the first and second frame elements 102a, 102 may facilitate the first and second bias-wrapped membrane 104, 312 conforming with each of the first and second frame elements 102a, 102b. One or both of the second pitch angle and the fourth pitch angle may be between approximately 15 degrees and approximately 85 degrees. In some embodiments. the pitch angle may range between approximately 0 degrees and 90 degrees. For example, the pitch angle may range between approximately 5 degrees and approximately 80 degrees, or between approximately 15 degrees and approximately 70 degrees, or between approximately 30 degrees and approximately 50 degrees. In certain instances, a single bias-wrapped membrane may be used.

FIG. 6A is a photograph of the implantable medical device 100 shown in FIGS. 4A and 5A in an example third step of a method of making the implantable medical device. FIG. 6B is a schematic side perspective view of the implantable medical device shown in FIG. 6A and FIG. 6B is a simplified cross-sectional view of the implantable medical device shown in FIG. 6B. A second flange 408 may be placed along second bias-wrapped membrane 312. As shown, the second bias-wrapped membrane 312 is everted (an airgap is shown between layers of the second bias-wrapped membrane 312 for illustrative purposes) over the flange 408 and the second frame element 102b is applied along the everted portion of the second bias-wrapped membrane 312. As shown in FIG. 7B, the first bias-wrapped membrane is everted over the second frame element 102b (and first frame element 102a). In certain instances, the second bias-wrapped membrane 312 may be everted in the first direction along the mandrel 400. In certain instances, the second bias-wrapped membrane 312 may be everted in the second direction along the mandrel 400 to sandwich the first frame element 102a and the second frame element 102b between the first bias-wrapped membrane 204 and the second bias-wrapped membrane 312. The second frame element 102b may be applied to the first side of the flange of the mandrel 400.

FIG. 7A is a photograph of the implantable medical device 100 shown in FIGS. 4A, 5A and 6A in an example fourth step of a method of making the implantable medical device 100. FIG. 7B is a schematic side perspective view of the implantable medical device 100 of FIG. 7A. In certain instances, the first bias-wrapped membrane 204 is everted over the second frame element 102b to sandwich the first frame element 102a and the second frame element 102b. FIG. 7B is a simplified cross-sectional illustration of the implantable medical device, shown in FIG. 7A, in accordance with an embodiment. As shown, the first bias-wrapped membrane 104 may be arranged on the first side 140 of the first frame element 102a and the first side 416 of the second frame element 102b and the second bias-wrapped membrane 312 may be arranged on the second side 412 of the first frame element 102a and the second side 414 of the second frame element 102b.

In certain instances, at least a portion of the first bias-wrapped membrane 104 may be bonded to at least a portion of the second bias-wrapped membrane 312. In addition, at least a portion of the first bias-wrapped membrane 104 may be bonded to at least a portion of the first frame element 102a and the second frame element 102b and at least a portion of the second bias-wrapped membrane 312 may be bonded to at least a portion of the first frame element 102a and the second frame element 102b.

While described above as having two flanges, the first flange 404 and second flange 408, the implantable medical device 100 may comprise a single flange or may comprise a plurality of flanges. In such embodiments, the implantable medical device 100 may act as an anchor or fixation device through the use of one or more flanges. As previously described, due to at least the one or more flanges being conformable to a flat and/or curved surface, the one or more flanges may be implemented in use with a flat and/or curved surface of varying organs, for example the stomach, heart and liver, or a blood vessel, thus coupling the implantable medical device 100 to the surface tissue of the desired organ and/or blood vessel.

The films of various embodiments described herein, which can be described as graft components, may be formed of a biocompatible material. In certain instances, such materials may include a fluoropolymer, such as a polytetrafluoroethylene (PTFE) polymer or an expanded polytetrafluoroethylene (ePTFE) polymer. In some instances, the graft may be formed of, such as, but not limited to, a polyester, a silicone, a urethane, a polyethylene terephthalate, or another biocompatible polymer, or combinations thereof. In some instances, bioresorbable or bioabsorbable materials may be used, for example a bioresorbable or bioabsorbable polymer. In some instances, the graft components, or film materials, can include Dacron, polyolefins, carboxy methylcellulose fabrics, polyurethanes, or other woven, non-woven, or film elastomers. In certain instances, the graft components may include polyethylene, such as an expanded polyethylene (ePE) that exhibits a similar (e.g., in structure and/or function) fibrillated or fibril structure as discussed for ePTFE.

In addition, nitinol (NiTi) may be used as the material of the frame (including any of the frames discussed herein), but other materials such as, but not limited to, stainless steel, L605 steel, polymers, MP35N steel, polymeric materials, Pyhnox, Elgiloy, or any other appropriate biocompatible material, and combinations thereof, can be used as frame material. The super-elastic properties and softness of NiTi may enhance the conformability of a frame. In addition, NiTi can be shape-set into a desired shape. That is, NiTi can be shape-set so that the frame tends to self-expand into a desired shape when a frame is unconstrained, such as when the frame is deployed out from a delivery system (not shown).

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. An implantable medical device comprising:
a frame including a first structural element having a first side and a second side opposite the first side; and
a bias-wrapped membrane, comprising at least a first helically wrapped film and a second helically wrapped film at least partially overlapping the first helically wrapped film at an angle, each of the first helically wrapped film and the second helically wrapped film arranged on the first side of the first structural element, and;
wherein the first helically wrapped film has a first plurality of fibrils having an arcuate shape extending along the first side of the first structural element, wherein the arcuate shape of the first plurality of fibrils extend for a majority of the first structural element side.

2. The implantable medical device of claim 1, wherein the frame includes a second structural element having a first side and a second side opposite the first side, wherein the second structural element has a circumference smaller than a circumference of the first frame element, wherein the first plurality of fibrils of the first helically wrapped film extend along the first side of the second structural element.

3. The implantable medical device of claim 2, wherein the second helically wrapped film extends along the second side of the first structural element and the second structural element, and wherein the second helically wrapped film includes a second plurality of fibrils along the second side of the first structural element with an arcuate shape and the second plurality of fibrils extend along the second side of the second structural element.

4. The implantable medical device of claim 3, wherein the frame includes a third structural element that extends at an angle of between approximately 15 degrees and approximately 85 degrees relative to a longitudinal axis of the frame, wherein the first plurality of fibrils extends along the third structural element side at an angle between approximately 15 degrees and approximately 85 degrees in a helical configuration.

5. The implantable medical device of claim 1, wherein at least a portion of the first helically wrapped film of the bias-wrapped membrane is coupled to at least part of the second helically wrapped film of the bias-wrapped membrane.

6. The implantable medical device of claim 1, wherein the first structural element is coupled to at least a portion of the bias-wrapped membrane along at least one of the first side and second side of the first structural element and wherein the second structural element is coupled to at least a portion of the bias-wrapped membrane along at least one of the first side and second side of the second structural element.

7. The implantable medical device of claim 1, wherein the first plurality of fibrils are configured to slide relative to adjacent arcuate fibrils.

8. The implantable medical device of claim 1, wherein the first structural element is a flange and wherein a sensor is coupled to or integral with the flange such that the flange is operable to engage the sensor and tissue for operable engagement between the sensor and the tissue.

9. The implantable medical device of claim 1, wherein the first structural element is a flange and the implantable medical device comprises an occluder coupled to or integral with the flange such that the flange is configured to anchor the occluder to tissue.

10. An implantable medical device comprising:
a frame including a first frame element having a first circumference and a second frame element having a second circumference, the first frame element having a first side and a second side opposite the first side;
a bias-wrapped membrane, comprising at least a first helically wrapped film and a second helically wrapped film at least partially overlapping the first helically wrapped film at an angle, wherein the bias-wrapped membrane is arranged on the first side of the first frame element and wherein the bias-wrapped membrane has a first plurality of fibrils arranged in an arcuate shape along the first side of the first frame element and a second plurality of fibrils arranged in an arcuate shape along the first side of the second frame element; and
wherein the bias-wrapped membrane has a third plurality of fibrils arranged between the first and second plurality of fibrils, wherein the third plurality of fibrils have a pitch angle.

11. A method of making an implantable medical device that includes a frame including a first frame element having a first side opposite a second side and a first diameter and a second frame element having a first side opposite a second side and having a second diameter, the first diameter being different than the second diameter, the method comprising:
elongating a first bias-wrapped membrane comprising a first helically wrapped film and second helically wrapped film overlapping the first helically wrapped film at an angle, so as to alter a first wrap angle of a first plurality of fibrils of the first bias-wrapped membrane;
arranging the elongated first bias-wrapped membrane on the first side of the first frame element and the second frame element so as to define a first-bias-wrapped membrane first edge along the first frame element and an arcuate shaped bias wrapped film edge along the first frame element;
elongating a second-bias wrapped membrane, comprising a first helically wrapped film and second helically wrapped film overlapping the first helically wrapped film at an angle, to alter a second wrap angle of a second plurality of fibrils of the second bias-wrapped membrane; and
arranging the elongated second-bias-wrapped membrane on the second side of the first frame element and the second frame element to define a second-bias-wrapped membrane first edge along the first frame element, wherein the second-bias-wrapped membrane first edge has an arcuate shape.

12. The method of claim 11, further comprising coupling at least a part of the first bias-wrapped membrane to at least a part of the second bias-wrapped membrane.

13. The method of claim 11, further comprising coupling at least a part of the first bias-wrapped membrane to at least a part of the first frame element and the second frame element, and coupling at least a part of the second bias-wrapped membrane to at least a part of the first frame element and the second frame element.

14. The method of claim 12, wherein the first plurality of fibrils are configured to slide relative to the second plurality of fibrils, and the first bias-wrapped membrane and the second bias-wrapped membrane are configured to maintain contact with the first frame element and the second frame element.

15. The method of claim 12, wherein the pitch angle of the first plurality of fibrils is a first pitch angle in the bias-wrapped membrane that is aligned with the first frame element and a second pitch angle in the bias-wrapped membrane that is aligned with the second frame element, wherein the first pitch angle is different than the second pitch angle.

16. The method of claim 15, wherein the pitch angle of the second plurality of fibrils includes a third pitch angle in the bias-wrapped membrane that is aligned with the first frame element and a fourth pitch angle in the bias-wrapped membrane that is aligned with the second frame element, wherein the third pitch angle is different than the fourth pitch angle.

17. The method of claim 16, wherein at least one of the second pitch angle and the fourth pitch angle is between approximately 15 degrees and approximately 85 degrees.

18-19. (canceled)
